(19) United States
(12) Patent Application Publication
(10) Pub. No.: US 2012/0267533 A1
(43) Pub. Date: Oct. 25, 2012

(54) PROCESSES FOR THE PREPARATION OF FORM I AND FORM II OF PALONOSETRON HYDROCHLORIDE

(75) Inventors: Gyanendra Pandey, Siddharthnagar (IN); Kaptan Singh, Ghaziabad (IN); Surender Dhingra, New Delhi (IN); Mohan Prasad, Gurgaon (IN); Tippasandra G. Chandrashekhar, Gurgaon (IN); Somenath Ganguly, Gurgaon (IN); Rita Santhakumar, Gurgaon (IN)

(73) Assignee: RANBAXY LABORATORIES LIMITED, New Delhi, Delhi (IN)

(21) Appl. No.: 13/379,898

(22) PCT Filed: Jun. 30, 2010
(86) PCT No.: PCT/IB2010/053010

§ 371 Date: Jul. 13, 2012

(30) Foreign Application Priority Data
Jun. 30, 2009 (IN) 1347/DEL/2009

(51) Int. Cl. C07D 453/02; G01J 5/02

(52) U.S. Cl. 250/339.08; 546/99

(57) ABSTRACT

The present invention relates to processes for the preparation of Form I and Form II of palonosetron hydrochloride. The present invention further relates to a method of determining the polymorphic forms of palonosetron hydrochloride using Fourier-Transform Infra-red (FTIR) method.

### FIELD OF THE INVENTION

The present invention relates to processes for the preparation of Form I and Form II of palonosetron hydrochloride. The present invention further relates to a method of determining the polymorphic forms of palonosetron hydrochloride using Fourier-Transform Infra-red (FTIR) method.

### BACKGROUND OF THE INVENTION

Palonosetron is chemically (3aS)-2-[(S)-1-Azabicyclo[2.2.2]oct-3-yl]-2,3,3a, 4,5,6-hexahydro-1-oxo-1H-benz[de]isoquinoline of Formula II having the structure as depicted below:

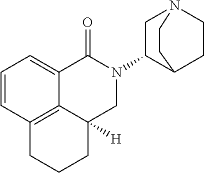

FORMULA II

Palonosetron is marketed in the form of its hydrochloride salt. Palonosetron is a 5-HT3 receptor antagonist and it is used for the treatment of nausea and vomiting often accompanying cancer chemotherapy.

U.S. Pat. No. 5,510,486 provides a process for preparing palonosetron hydrochloride, wherein the final recrystallization is carried out from isopropanol and water. PCT Publication No. WO 2008/073757 says that a mixture of Form I and Form II of palonosetron hydrochloride is obtained as per the method described in U.S. Pat. No. 5,510,486. According to PCT Publication No. WO 2008/073757, Form I of palonosetron hydrochloride is prepared by crystallization of palonosetron hydrochloride from an ethanolic solution held at ambient temperature for one week and Form II is prepared by crystallization of palonosetron hydrochloride from a hot ethanolic solution by immediate filtration upon cooling to room temperature. PCT Publication No. WO 2008/073757 also provides a process for preparing amorphous palonosetron hydrochloride by lyophilization. PCT Publication No. WO 2008/073757 further says that Form I and Form II can be distinguished from one another by X-ray powder diffraction patterns, thermal properties, purity and methods of manufacture.

PCT Publication No. WO 2008/051564 provides processes for preparing two crystalline forms of palonosetron hydrochloride and distinguishes them with a set of X-ray powder diffraction peaks at about 12.1, 15.8 and 17.3 and at about 13.0, 15.4, and 17.5, respectively. These crystalline forms are prepared from a mixture of isopropanol and water and the formation of the crystalline forms is controlled by cooling rate.

U.S. Patent Publication No. 2008/0058367 provides a process for the preparation of a crystalline form of palonosetron hydrochloride, which is characterized by X-ray powder diffraction peaks at 7.1, 13.8, 14.2, 15.8, 18.5, 19.7, 20.0 and 24.4±0.2 degrees 2 theta. The process provided in U.S. Patent Publication No. 2008/0058367 involves the crystallization of palonosetron hydrochloride from methanol solvent system. The method involves dissolving palonosetron hydrochloride in methanol at about 50° C. to about 60° C. followed by distillation of methanol and cooling the resultant solution to about 0° to about 5° C. followed by stirring for about 4 hours. The solid is separated and washed with methanol at about 45° C. to about 50° C. to obtain palonosetron hydrochloride having X-ray powder diffraction peaks at 7.1, 13.8, 14.2, 15.8, 18.5, 19.7, 20.0 and 24.4±0.2 degrees 2 theta.

### SUMMARY OF THE INVENTION

The present inventors have found that the quantity of water present in the polar organic solvents during the crystallization of palonosetron hydrochloride affects the polymorphic integrity of palonosetron hydrochloride. The present inventors have also found that Form I and Form II of palonosetron hydrochloride can be selectively obtained by controlling quantity of water present in the polar organic solvents and/or by using nonpolar solvents. By employing the processes of the present invention, Form I or Form II palonosetron hydrochloride can be prepared selectively in a consistent and reproducible manner. The present inventors have also developed a simple and efficient method for determining the polymorphic forms of palonosetron hydrochloride using Fourier-Transform Infra-red (FTIR) method. The method can be used to effectively determine whether a sample of palonosetron hydrochloride is Form I or Form II, or a mixture thereof.

### BRIEF DESCRIPTION OF THE DRAWINGS

FIG. 1 depicts the X-Ray Powder Diffractogram (XRPD) of Form I of palonosetron hydrochloride.

Table 1 provides a table of the XRPD peaks of Form I of palonosetron hydrochloride as depicted in FIG. 1.

FIG. 2 depicts the Fourier-Transform Infra-red (FTIR) spectrum of Form I of palonosetron hydrochloride.

FIG. 3 depicts the Differential Scanning calorimetry (DSC) thermogram of Form I of palonosetron hydrochloride.

FIG. 4 depicts the XRPD of Form II of palonosetron hydrochloride.

Table 2 provides a table of the XRPD peaks of Form II of palonosetron hydrochloride as depicted in FIG. 4.

FIG. 5 depicts the FTIR spectrum of Form II of palonosetron hydrochloride.

FIG. 6 depicts the DSC of Form II of palonosetron hydrochloride.

FIG. 7 depicts the XRPD of the mixture of Form I and Form II of palonosetron hydrochloride.

FIG. 8 depicts comparative FTIR spectra of Form I, Form II, and the mixture of Form I and Form II of palonosetron hydrochloride.

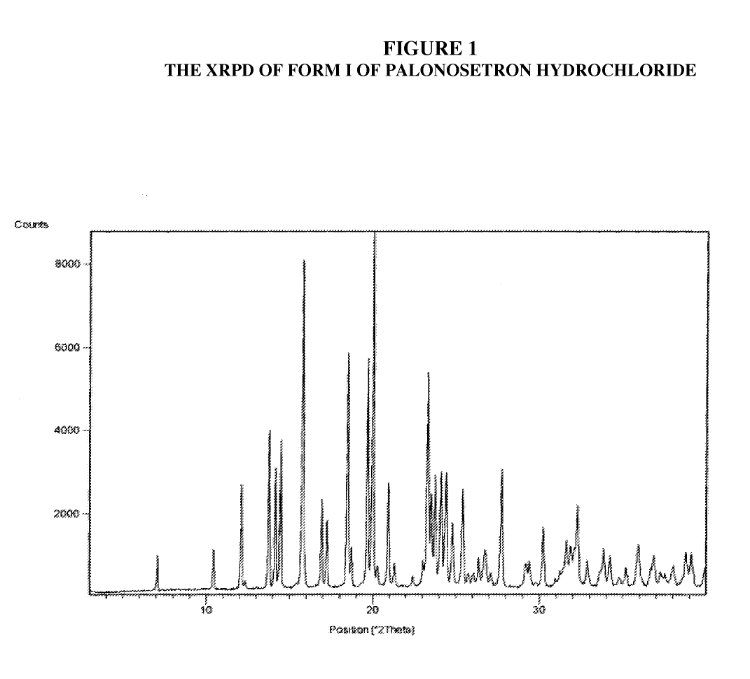

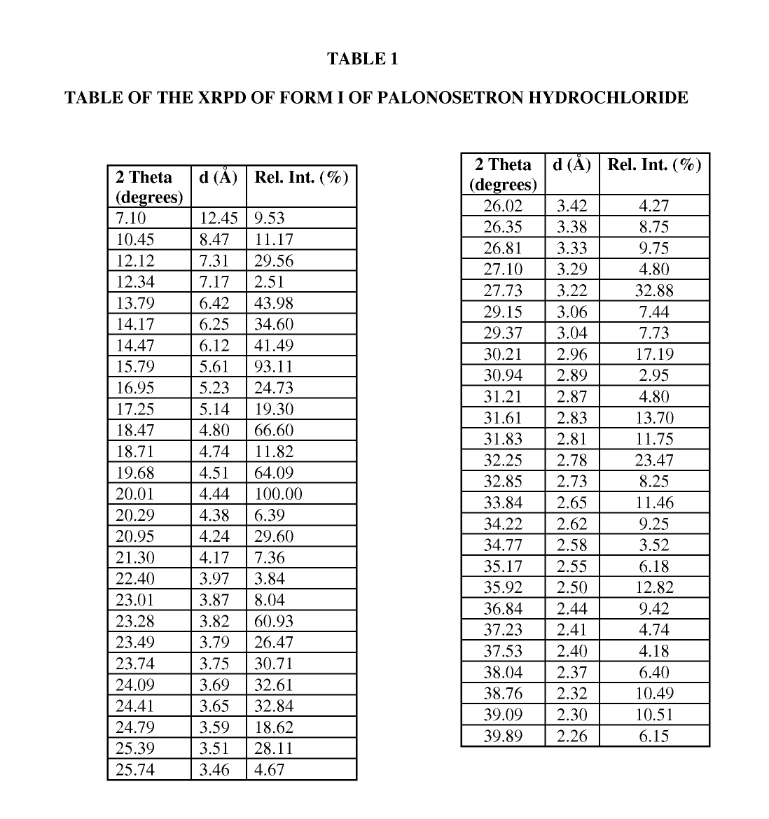

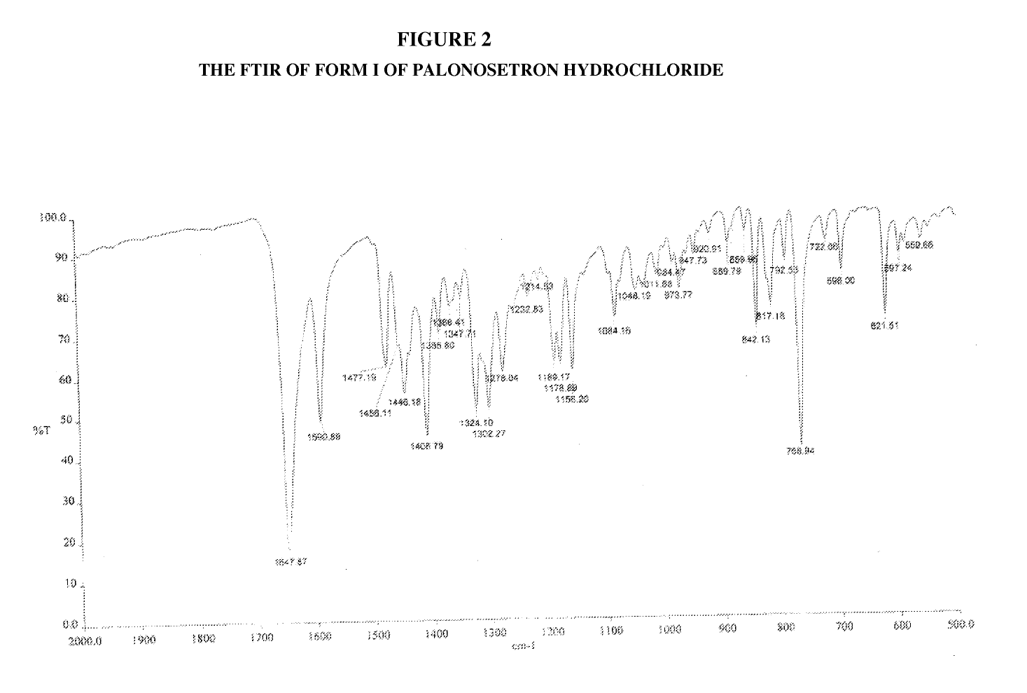

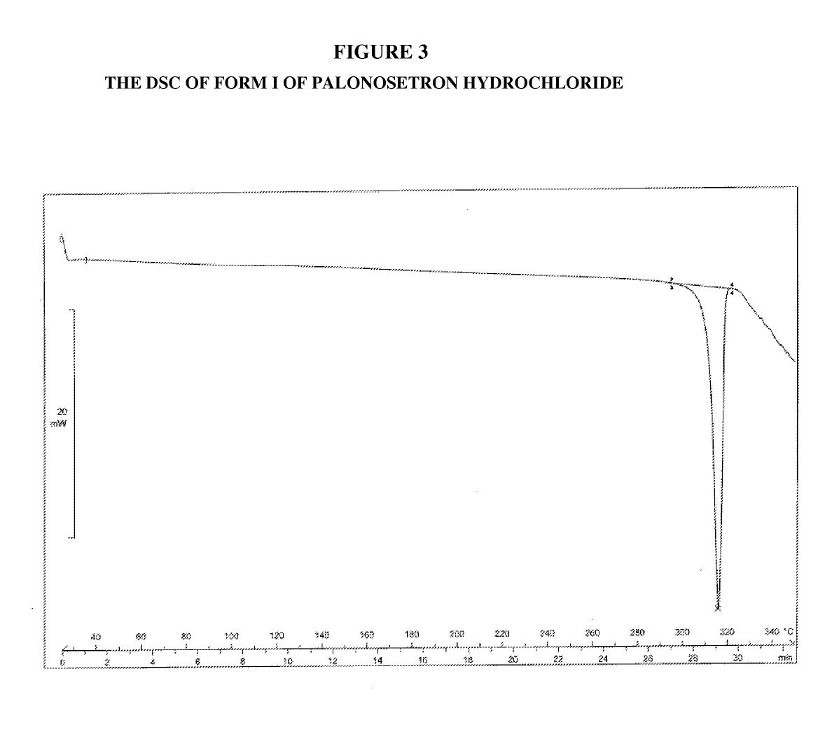

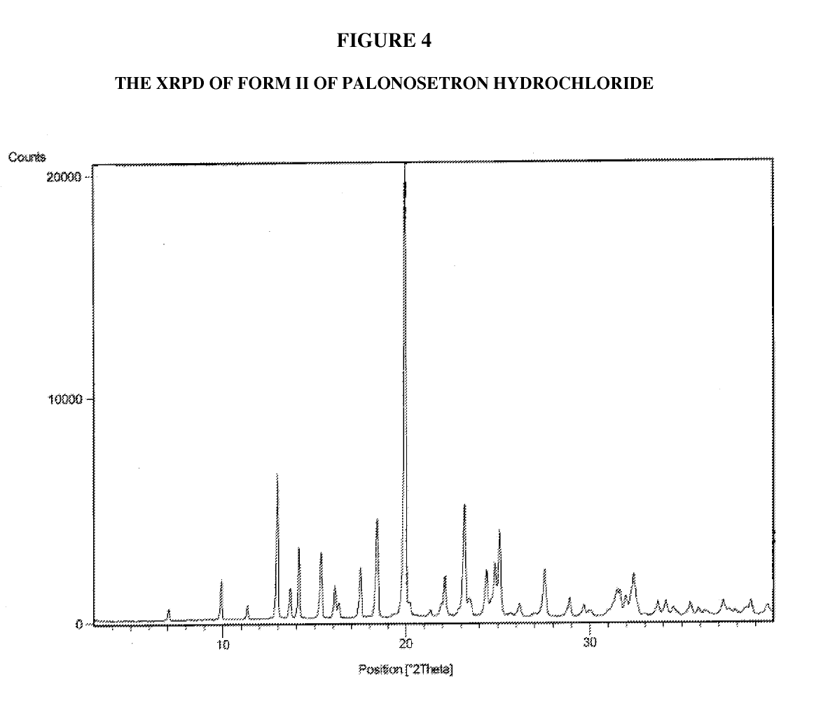

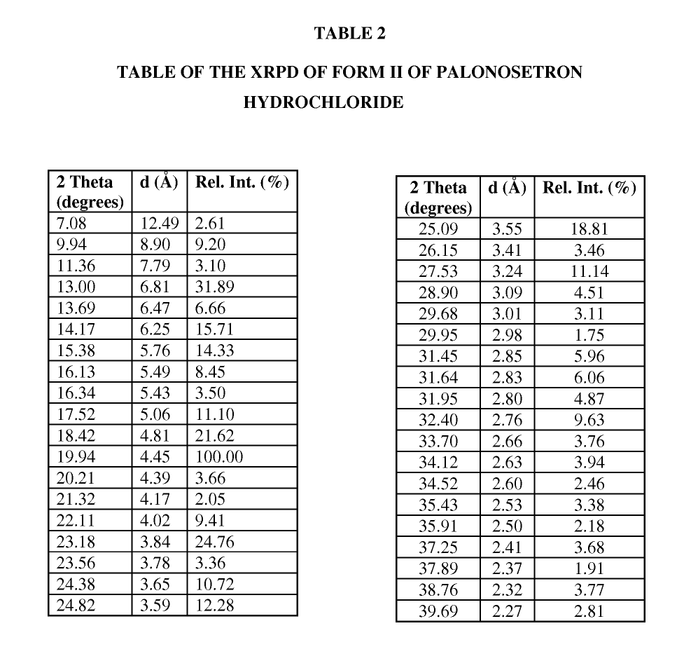

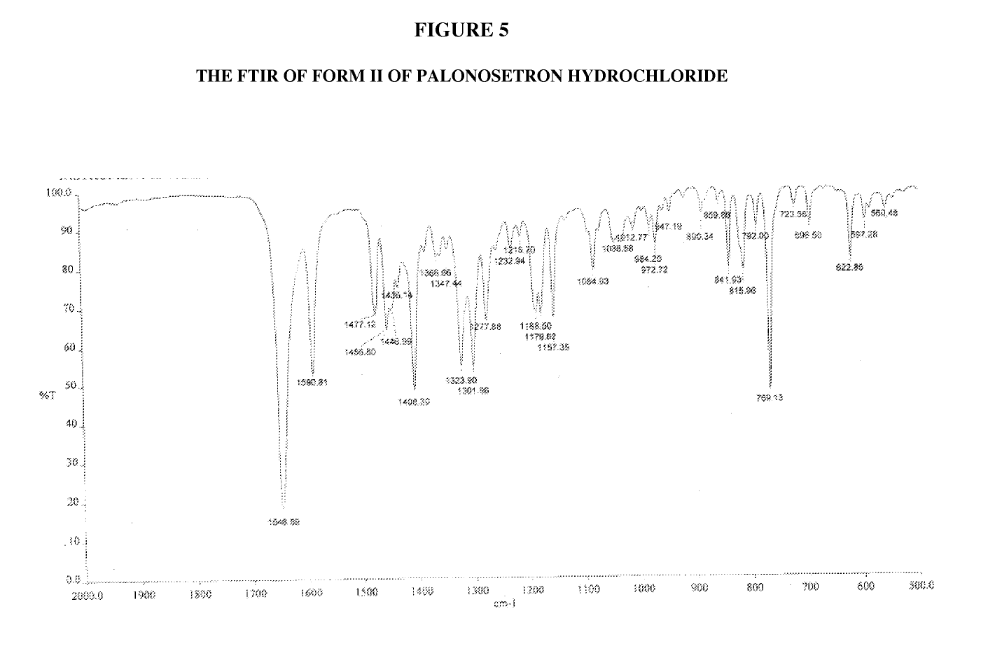

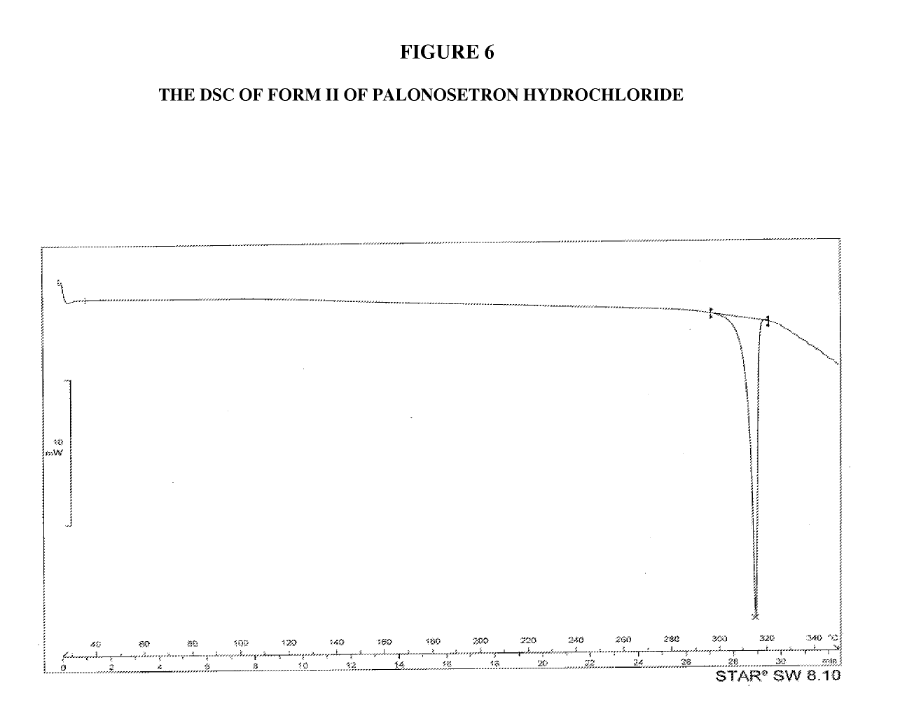

### DETAILED DESCRIPTION OF THE INVENTION

A first aspect of the invention provides a process for the preparation of the Form I of palonosetron hydrochloride, wherein the process comprises:

a) treating palonosetron hydrochloride with a polar organic solvent and water, wherein the quantity of water is 45% or more to the weight of palonosetron hydrochloride; and

b) isolating the Form I of palonosetron hydrochloride from the mixture thereof.

Palonosetron hydrochloride used as a starting material may be obtained according to the methods provided in the prior art, for example, U.S. Pat. Nos. 5,202,333; 5,567,818; and 5,510,486; J. Med. Chem., 36, p. 2645-2657 (1993), or Organic Process Research & Development, 1, p. 117-120 (1997). The palonosetron hydrochloride is treated with a polar organic solvent and water. The water is used in a quantity of about 45% or more to the weight of palonosetron hydrochloride. The quantity of water may be, for example, from about 47% to about 52% to the weight of palonosetron hydrochloride. The polar organic solvent may be a C₁₋₃ alkanol, for example, ethanol. The treatment of palonosetron hydrochloride with the polar organic solvent and water may be carried out by dissolving palonosetron hydrochloride in the polar organic solvent by heating and subsequently treating with water at the hot condition. The solution obtained is cooled to about 25° C. or below and stirred for sufficient time to obtain Form I of palonosetron hydrochloride. The Form I of palonosetron hydrochloride may be isolated from the mixture by filtration, distillation, decantation, concentration, or a combination thereof.

A second aspect of the present invention provides a process for the preparation of the Form I of palonosetron hydrochloride, wherein the process comprises:

a) dissolving palonosetron hydrochloride in water;

b) removing the water from the solution obtained in step a);

c) treating the residue obtained in step b) with a polar organic solvent substantially free of water at a temperature of about 25° C. or below; and

d) isolating Form I of palonosetron hydrochloride from the mixture thereof.

Palonosetron hydrochloride used as a starting material may be obtained according to the methods provided in the prior art, for example, U.S. Pat. Nos. 5,202,333; 5,567,818; and 5,510,486; J. Med. Chem., 36, p. 2645-2657 (1993), or Organic Process Research & Development, 1, p. 117-120 (1997). The palonosetron hydrochloride is dissolved in water. The dissolution process may be effected by stirring and/or by heating. The water is subsequently removed from the solution to obtain a substantially dry residue. The substantially dry residue obtained is treated with a polar organic solvent substantially free of water. The polar organic solvent may be a C₁₋₃ alkanol, for example, absolute ethanol. The reaction mixture obtained is stirred at a temperature of about 25° C. or below for sufficient time to obtain Form I of palonosetron hydrochloride. The Form I of palonosetron hydrochloride may be isolated from the mixture by filtration, distillation, decantation, concentration, or a combination thereof.

A third aspect of the present invention provides a process for the preparation of the Form II of palonosetron hydrochloride, wherein the process comprises:

a) dissolving palonosetron hydrochloride in a polar organic solvent;

b) treating the solution obtained in step a) with a non-polar organic solvent; and

c) isolating Form II of palonosetron hydrochloride from the mixture thereof.

Palonosetron hydrochloride used as a starting material may be obtained according to the methods provided in the prior art, for example, U.S. Pat. Nos. 5,202,333; 5,567,818; and 5,510,486; J. Med. Chem., 36, p. 2645-2657 (1993), or Organic Process Research & Development, 1, p. 117-120 (1997). The palonosetron hydrochloride is dissolved in a polar organic solvent. The polar organic solvent may be a C₁₋₃ alkanol, for example, methanol. The dissolution may be effected by heating the reaction mixture to a temperature of about 60° C. or above. The solution obtained is treated with a non-polar organic solvent. The non-polar organic solvent may be selected from a group consisting of ethyl acetate, toluene, pentane, hexane, octane, diethyl ether, methyl t-butyl ether, cyclohexane, and petroleum ether. The mixture obtained is stirred at a temperature of about 25° C. or below for sufficient time to obtain Form II of palonosetron hydrochloride. The Form II of palonosetron hydrochloride may be isolated from the mixture by filtration, distillation, decantation, concentration, or a combination thereof.

A fourth aspect of the present invention provides a method of determining the polymorphic form of palonosetron hydrochloride, wherein the method comprises:

a) generating an FTIR spectrum of a sample of palonosetron hydrochloride; and

b) determining the polymorphic form of said sample by the ratio of intensity of the peaks at about 1456, about 1446 and about 1408 cm⁻¹.

The FTIR spectrum of the sample of palonosetron hydrochloride may be generated by using potassium bromide pellet, according the general test methods provided in the USP 25, page 1920. Commercially available instruments, for example, Perkin Elmer Spectrum One instrument, may be used for the purposes.

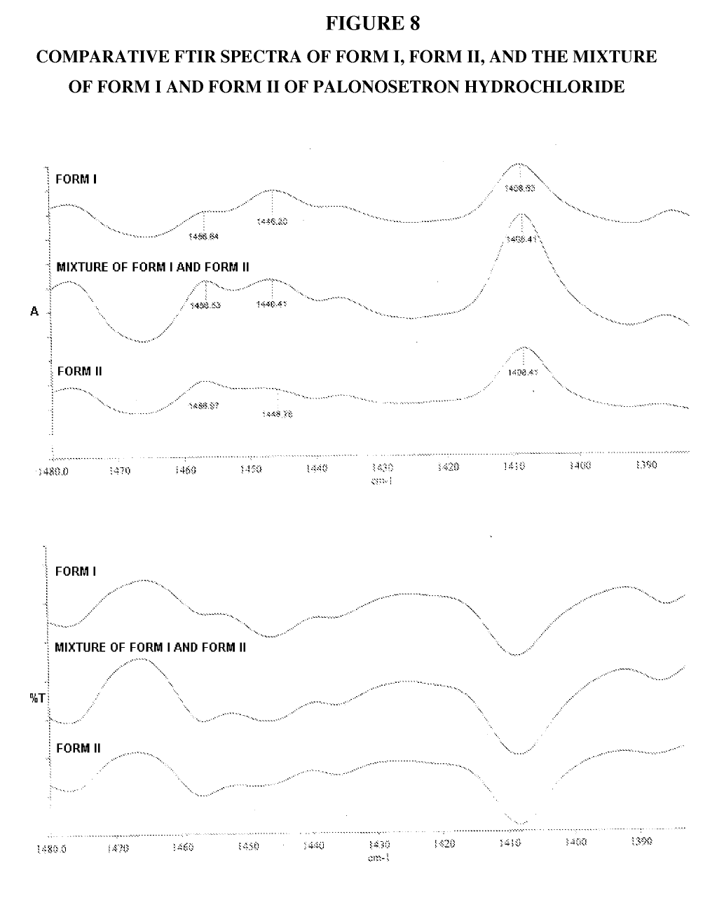

The sample of palonosetron hydrochloride is determined as Form I or as Form II by the ratio of intensity of the peaks at about 1456, about 1446 and about 1408 cm⁻¹. The ratio of the intensity of the peak at about 1446 cm⁻¹ to that of the peak at about 1408 cm⁻¹ is equal to or greater than about 0.32 in Form I as depicted in FIG. 8 of the accompanied drawing. The ratio of the intensity of the peak at about 1446 cm⁻¹ to that of the peak at about 1408 cm⁻¹ is equal to or less than about 0.07 in Form II as depicted in FIG. 8 of the accompanied drawing.

The ratio of the intensity of the peak at about 1446 cm⁻¹ to that of the peak at about 1456 cm⁻¹ is equal to or greater than about 3 in Form I as depicted in FIG. 8 of the accompanied drawing. The ratio of the intensity of the peak at about 1446 cm⁻¹ to that of the peak at about 1456 cm⁻¹ is equal to or less than about 0.25 in Form II as depicted in FIG. 8 of the accompanied drawing.

The ratio of the intensity of the peak at about 1456 cm⁻¹ to that of the peak at about 1408 cm⁻¹ is equal to or less than about 0.11 in Form I as depicted in FIG. 8 of the accompanied drawing. The ratio of the intensity of the peak at about 1456 cm⁻¹ to that of the peak at about 1408 cm⁻¹ is equal to or greater than about 0.29 in Form II as depicted in FIG. 8 of the accompanied drawing.

The ratio of the intensity of the peak at about 1456 cm⁻¹ to that of the peak at about 1446 cm⁻¹ is equal to or less than about 0.35 in Form I as depicted in FIG. 8 of the accompanied drawing. The ratio of the intensity of the peak at about 1456 cm⁻¹ to that of the peak at about 1446 cm⁻¹ is equal to or greater than about 4.2 in Form II as depicted in FIG. 8 of the accompanied drawing.

Powder XRD of the samples were determined by using Panalytical X'Pert Pro X-Ray Powder Diffractometer in the range 3 to 40 degree 2 theta and under tube voltage and current of 45 Kv and 40 mA respectively. Copper radiation of wavelength 1.54 angstrom and Xceletor detector was used.

DSC thermograms were recorded using Mettler DSC 821 instrument. About 3 to 5 mg of sample was scanned from 25° C. to 350° C. at a heating rate of 10° C/min under nitrogen flow of 20 ml/min using alumina crucibles covered with lid having one hole.

While the present invention has been described in terms of its specific embodiments, certain modifications and equivalents will be apparent to those skilled in the art and are intended to be included within the scope of the present invention.

### Example 1

### Preparation of Form I of Palonosetron Hydrochloride

Absolute ethanol (500 ml) and palonosetron hydrochloride (HPLC Purity 98%, 50 g) were added together at about 25° C. The mixture was heated at 75° C. to 78° C. and deionised water (24 g) was added to obtain a clear solution. Activated carbon (5 g) was added to the solution and stirred at the same temperature for 10 to 15 minutes. The mixture was filtered at the hot condition through Celite bed, washed with hot ethanol (25 ml) and cooled to about 25° C. The reaction mixture obtained was stirred at about 25° C. for 2 hours, filtered and washed with cold absolute ethanol (25 ml). The solid product was dried under vacuum (680 to 710 mmHg) at 40° C. to 45° C. for 12 hours to obtain the title compound.

Yield: 33 g

HPLC Purity: 99.9%

### Example 2

### Preparation of Form I of Palonosetron Hydrochloride

Deionised water (5.0 ml) and palonosetron hydrochloride (1 g) were added together at about 25° C. The mixture was stirred at about 25° C. for 10 minutes to obtain a clear solution. The water was recovered completely under vacuum at 45° C. to get a solid residue. Absolute ethanol (5 ml) was added to the residue and stirred at about 25° C. for 15 minutes. The mixture was filtered, washed with cold absolute ethanol (2 ml) and dried under vacuum at 45° C. to obtain the title compound.

Yield: 0.8 g

### Example 3

### Preparation of Form II of Palonosetron Hydrochloride

Methanol (8 ml) and palonosetron hydrochloride (1 g) were added together at about 25° C. The mixture was heated at 65° C. to obtain a solution. Ethyl acetate (30 ml) was added slowly to the solution at 60° C. to 65° C. The mixture was cooled to about 25° C. and stirred for 1 hour. The mixture was filtered, washed with ethyl acetate (2 ml) and dried under vacuum at 45° C. to obtain the title compound.

Yield: 0.9 g

### Comparative Example

### Preparation of a Mixture of Forms I and II of Palonosetron Hydrochloride

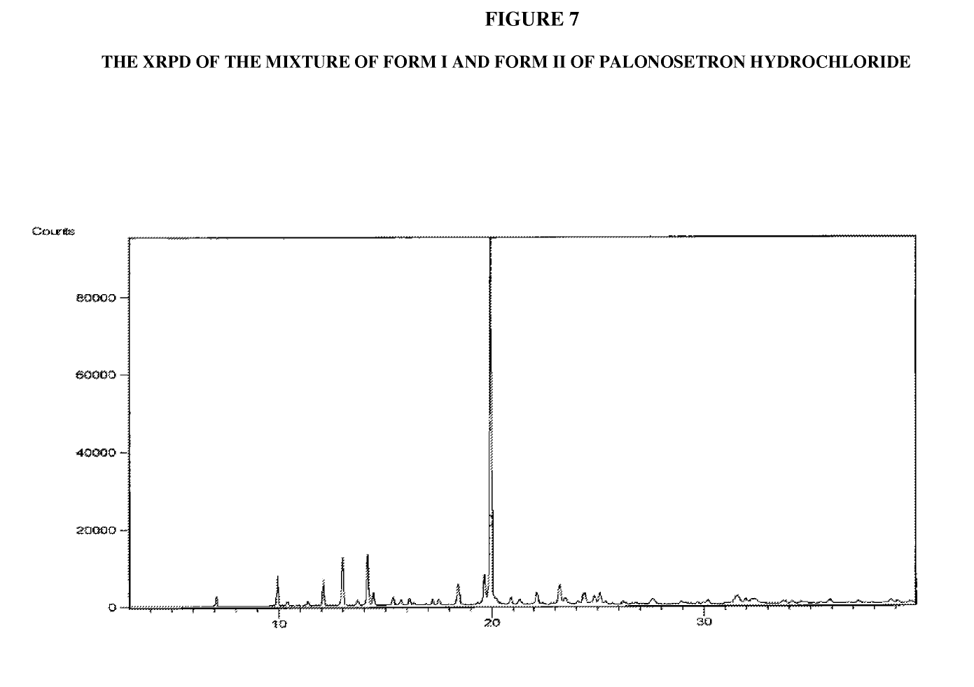

Absolute ethanol (500 ml) and palonosetron hydrochloride (50 g) were added together at about 25° C. The mixture was heated at 75° C. to 78° C. and deionised water (18 ml) was added to obtain a clear solution. Activated carbon (5 g) was added to the solution and stirred at the same temperature for 10 to 15 minutes. The mixture was filtered at the hot condition through a Celite bed, washed with hot ethanol (25 ml) and cooled to about 25° C. The reaction mixture obtained was stirred at about 25° C. for 2 hours, filtered and washed with cold absolute ethanol (25 ml). The solid product was dried under vacuum (680 to 710 mmHg) at 40° C. to 45° C. for 12 hours to obtain the title compound having an XRPD pattern as depicted in FIG. 7 of the accompanied drawing.

Yield: 33 g

We claim:

1. A process for the preparation of the Form I of palonosetron hydrochloride, wherein said process comprises:
a) treating palonosetron hydrochloride with a polar organic solvent and water, wherein the quantity of water is about 45% or more to the weight of palonosetron hydrochloride; and
b) isolating the Form I of palonosetron hydrochloride from the mixture thereof.

2. A process for the preparation of the Form I of palonosetron hydrochloride, wherein the process comprises:
a) dissolving palonosetron hydrochloride in water;
b) removing the water from the solution obtained in step a);
c) treating the residue obtained in step b) with a polar organic solvent substantially free of water at a temperature of about 25° C. or below; and
d) isolating Form I of palonosetron hydrochloride from the mixture thereof.

3. A process for the preparation of the Form II of palonosetron hydrochloride, wherein the process comprises:
a) dissolving palonosetron hydrochloride in a polar organic solvent;
b) treating the solution obtained in step a) with a non-polar organic solvent; and
c) isolating Form II of palonosetron hydrochloride from the mixture thereof.

4. A process according to claim 1, 2 or 3, wherein the polar organic solvent is a C₁₋₃ alkanol.

5. A process according to claim 3, wherein the non-polar organic solvent is selected from the group consisting of ethyl acetate, toluene, pentane, hexane, octane, diethyl ether, methyl t-butyl ether, cyclohexane, and petroleum ether.

6. A method of determining the polymorphic form of palonosetron hydrochloride, wherein the method comprises:
a) generating an FTIR spectrum of a sample of palonosetron hydrochloride; and
b) determining the polymorphic form of the sample by the ratio of intensity of the peaks at about 1456, about 1446, and about 1408 cm⁻¹.

7. A method according to claim 6, wherein the ratio of the intensity of the peak at about 1446 cm⁻¹to that of the peak at about 1408 cm⁻¹ is equal to or greater than about 0.32 in Form I.

8. A method according to claim 6, wherein the ratio of the intensity of the peak at about 1446 cm⁻¹ to that of the peak at about 1408 cm⁻¹ is equal to or less than about 0.07 in Form II.

9. A method according to claim 6, wherein the ratio of the intensity of the peak at about 1446 cm⁻¹ to that of the peak at about 1456 cm⁻¹ is equal to or greater than about 3 in Form I.

10. A method according to claim 6, wherein the ratio of the intensity of the peak at about 1446 cm⁻¹ to that of the peak at about 1456 cm⁻¹ is equal to or less than about 0.25 in Form II.

11. A method according to claim 6, wherein the ratio of the intensity of the peak at about 1456 cm⁻¹ to that of the peak at about 1408 cm⁻¹ is equal to or less than about 0.11 in Form I.

12. A method according to claim 6, wherein the ratio of the intensity of the peak at about 1456 cm⁻¹ to that of the peak at about 1408 cm⁻¹ is equal to or greater than about 0.29 in Form II.

13. A method according to claim 6, wherein the ratio of the intensity of the peak at about 1456 cm⁻¹ to that of the peak at about 1446 cm⁻¹ is equal to or less than about 0.35 in Form I.

14. A method according to claim 6, wherein the ratio of the intensity of the peak at about 1456 cm⁻¹ to that of the peak at about 1446 cm⁻¹ is equal to or greater than about 4.2 in Form II.
